(19) United States
(12) Patent Application Publication
(10) Pub. No.: US 2004/0001883 A1
(43) Pub. Date: Jan. 1, 2004

(54) Meloxicam suppositories

(75) Inventors: Mayumi Matsui, Ikeda (JP); Toshimitsu Ohki, Ikeda (JP); Koichi Wada, Inagawa-cho (JP)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/394,088

(22) Filed: Mar. 21, 2003

Related U.S. Application Data
(60) Provisional application No. 60/390,458, filed on Jun. 20, 2002.

(30) Foreign Application Priority Data
Mar. 30, 2002 (EP) 02007455.5

(51) Int. Cl. A61K 31/542; A61K 9/48

(52) U.S. Cl. 424/452; 514/224.2

(57) ABSTRACT

The present invention relates to suppositories consisting essentially of the active ingredient meloxicam or a pharmaceutically acceptable salt thereof and at least one pharmaceutically acceptable excipient.

### RELATED APPLICATIONS

The benefit of United States provisional application Serial No. 60/390,458, filed Jun. 20, 2002 is hereby claimed.

### BACKGROUND OF THE INVENTION

The present invention relates to a suppository consisting essentially of the active ingredient meloxicam or a pharmaceutically acceptable salt thereof and at least one pharmaceutically acceptable excipient.

Meloxicam [2H-1,2-benzothiazine-3-carboxamide, 4-hydroxy-2-methyl-N-(5-methyl-2-thiazolyl)-1,1-dioxide] is a non-steroidal anti-inflammatory drug (NSAID) with anti-inflammatory, antipyretic and analgesic activity.

Rectal absorption of meloxicam has been reported (Arzneimittelforschung 47(3):253, 1997). The type and nature of a suppository base and a solubilizer affect the stability, release and absorption of a drug.

The formulation of meloxicam suppositories is described in J. Pharm. Sci. (Vol. 23:11, June 1999), wherein meloxicam is formulated in different suppository bases e.g. Witepsol H15 in combination with PVP K-90. The drug release of this formulation is comparable to commercially available meloxicam suppositories.

### SUMMARY OF THE INVENTION

The present invention relates to suppositories consisting essentially of the active ingredient meloxicam or a pharmaceutically acceptable salt thereof and at least one pharmaceutically acceptable excipient.

### BRIEF DESCRIPTION OF THE DRAWING

FIG. 1 shows plasma concentration profiles after rectal administration in beagle dogs of Formulation A ( [symbol] ), Formulation B ( [symbol] ), Formulation C (— — —), or the fat-based meloxicam suppository (- - -). The compositions of the formulations are described in Table 1.

### DESCRIPTION OF THE INVENTION

The problem underlying the present invention is to provide a meloxicam suppository, which shows a fast onset of meloxicam absorption and a high bioavailability of meloxicam.

Thus it has surprisingly been found that the problem underlying the invention is solved by a suppository containing a composition, consisting essentially of meloxicam or a pharmaceutically acceptable salt thereof, and at least one pharmaceutically acceptable excipient, wherein at least one of the excipients is a polyalkylene glycol.

According to the invention the term pharmaceutically acceptable salt stands for a meloxicam salt of an organic or inorganic base, as for example the meglumine, sodium, potassium or ammonium salt.

The polyalkylene glycols are as a rule polyethylene glycols, polypropylene glycols, or mixtures thereof, preferably polyethylene glycols.

The polyethylene glycol component typically has an average molecular weight from about 200 to about 6000. Commercially available polyethylene glycol materials, for example, from Nippon Yushi Co., Ltd., Tokyo, Japan, include e.g. PEG 400, PEG 800, PEG 1000, PEG 2000, PEG 3000, PEG 4000 and PEG 6000.

In a preferred embodiment the present invention relates to a suppository, wherein the suppository is a soft gelatin capsule suitable for rectal administration.

In a further preferred embodiment the present invention relates to a suppository wherein the composition comprises the meloxicam meglumine salt.

In another preferred embodiment the present invention relates to a suppository wherein the composition comprises the free meloxicam base.

In a particularly preferred embodiment the present invention relates to a suppository, wherein the polyalkylene glycol is selected from the group consisting of: polyethylene glycol 400, polyethylene glycol 4000 and polyethylene glycol 6000.

In another particularly preferred embodiment the present invention relates to a suppository which contains at least 0.1% by weight, preferably 0.1% to 5%, more preferably about 0.3% of a solubilizer or a mixture of solubilizers of the overall weight of said composition.

According to the invention solubilizers are preferably selected from the group consisting of: sodium hydroxide, potassium hydroxide, sodium bicarbonate, sodium citrate, and meglumine.

In a further particularly preferred embodiment the present invention relates to a suppository, wherein the composition contains 1 to 20 μg, preferably 3 to 15 μg, more preferably about 5 to 10 μg, of the free meloxicam base per mg of said composition.

In another particularly preferred embodiment the present invention relates to a suppository which contains at least 0.2 mg, preferably 0.2 to 5 mg, more preferably about 1 mg, of the solubilizer or mixture of solubilizers per mg of the free meloxicam base.

More particularly preferred according to the present invention is a suppository, which contains a composition consisting of:

a) 1 to 20 mg, preferably 3 to 15 mg, more preferably about 5 to 10 mg, of meloxicam;

b) 500 to 1500 mg, preferably 800 to 1200 mg, more preferably about 1000 mg, of one or more polyalkylene glycols selected from the group consisting of: polyethylene glycol 400, polyethylene glycol 4000 and polyethylene glycol 6000; and optionally

c) one or two additional excipients, e.g. a solubilizer, preferably meglumine or a stabilizer.

As a rule according to the invention a method for preparation of said suppository comprises the steps of:

a) intimately mixing meloxicam or a pharmaceutically acceptable salt thereof with the melted solubilizer; and

b) encapsulating or molding the composition obtained in step a) by a conventional method, for example, described in “Suppositories—From Dosage Forms to Clinical Applications—”, Nanzando Co., Ltd. 1985.

The suppository of the present invention is used for the preparation of a medicament with enhanced bioavailability for the treatment or prevention of polyarthritis, rheumatoid arthritis or inflammatory diseases.

The invention is further illustrated by the examples of suppository compositions given in Table 1. The invention should not be limited to these examples. According to the present invention the compositions B and C are manufactured into suppositories by conventional methods, which are described for instance in “Suppositories—From Dosage Forms to Clinical Applications—”, Nanzando co., Ltd. 1985.

A suitable method of manufacturing a composition according to the invention is e.g. to dispense each component of the composition in the suppository base, e.g. a polyethylene glycol. Subsequently all components are mixed in a suitable vessel, such as a stainless steel beaker, at room temperature. The mixture is stirred for about 0.5 hours, until a clear solution is obtained.

TABLE 1
Compositions of meloxicam for suppositories
Values are given in mg.

| | Formulation | | |
|---|---|---|---|
| | A | B | C |
| | Rectal | Conventional | Conventional |
| Dosage form | capsule | suppository | suppository |
| Meloxicam | 5.0 | 5.0 | 5.0 |
| Meglumine | 2.8 | — | — |
| Polyethylene glycol 400 | 992.2 | — | 250 |
| Polyethylene glycol 4000 | — | 905 | 655 |
| Polyethylene glycol 6000 | — | 90 | 90 |
| Total weight | 1000 | 1000 | 1000 |

Bioavailability tests have been conducted with suppository formulations according to the present invention and with a commercially available meloxicam formulation. The composition of the commercially available meloxicam suppository formulation is based on fats such as Suppocire® BP. The plasma concentration test was conducted using beagle dogs. Six to nine suppositories were tested for each batch.

The suppositories of the present invention were prepared according to the compositions shown in Table 1.

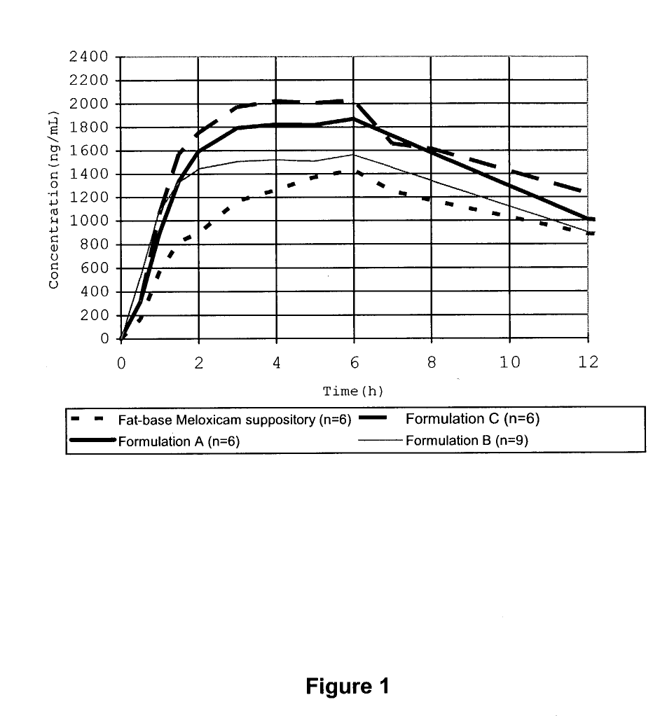

As shown in FIG. 1, the compositions according to the invention were found to be superior to the fat based composition with regard to the onset of meloxicam absorption and the bioavailability of meloxicam.

Stability tests were conducted with formulation type A at 30° C., 70% room humidity over six months in a PVC/aluminum pillow. Details of the stability test are shown in Table 2.

TABLE 2
Stability Test

| Storage time: 6 months | | Sample No. 1 | Sample No. 2 |
|---|---|---|---|
| Meloxicam content | | 99.3% | 100.4% |
| impurities | 2-amino-5-methyl | 0.24% | 0.15% |
| | thiazole | | |
| | Others | 0.25% | 0.10% |
| | Total | 0.49% | 0.25% |

In both samples the meloxicam content and the packaging material were found to be unchanged.

Included herein are exemplified embodiments, which are intended as illustrations of single aspects of the invention. Indeed, various modifications of the invention in addition to those herein will become apparent to those skilled in the art from the foregoing description and drawings. Such modifications are intended to fall within the scope of the present invention.

All publications and patent applications cited herein are incorporated by reference in their entireties.

What is claimed is:

1. A suppository containing a composition consisting essentially of meloxicam or a pharmaceutically acceptable salt thereof and at least one pharmaceutically acceptable excipient, wherein at least one of the excipients is a polyalkylene glycol.

2. The suppository capsule according to claim 1, wherein the suppository is a soft gelatin capsule suitable for rectal administration.

3. The suppository capsule according to claim 1 or 2, wherein the meloxicam or a pharmaceutically acceptable salt thereof comprises a meloxicam meglumine salt.

4. The suppository capsule according to claim 1 or 2, wherein the meloxicam or a pharmaceutically acceptable salt thereof comprises a free meloxicam base.

5. The suppository according to claim 1 or 2, wherein the polyalkylene glycol is selected from the group consisting of: polyethylene glycol 400, polyethylene glycol 4000 and polyethylene glycol 6000.

6. The suppository according to claim 1 wherein the composition comprises at least 0.1% by weight of the overall weight of said composition of a solubilizer or a mixture of solubilizers.

7. The suppository according to claim 4 wherein the composition comprises 1 to 20 μg of the free meloxicam base per mg of the composition.

8. The suppository according to claim 4 wherein the composition comprises at least 0.2 mg of the solubilizer or mixture of solubilizers per mg of the free meloxicam base.

9. A suppository which contains a composition consisting of:
a) 1 to 20 mg of meloxicam,
b) 500 to 1500 mg of one or more polyalkylene glycols selected from the group consisting of: polyethylene glycol 400, polyethylene glycol 4000 and polyethylene glycol 6000; and optionally
c) one or two additional excipients.

10. A method for the preparation of a suppository according to claim 1, which comprises the steps of:
a) intimately mixing meloxicam or a pharmaceutically acceptable salt thereof with a melted solubilizer; and
b) encapsulating or molding the composition obtained in step a).

11. A method for the treatment or prevention of polyarthritis, rheumatoid arthritis or inflammation diseases comprising administering a suppository according to claim 1 to a patient in need thereof.
